(19) United States
(12) Patent Application Publication
(10) Pub. No.: US 2023/0414069 A1
(43) Pub. Date: Dec. 28, 2023

(54) MEDICAL SUPPORT SYSTEM AND MEDICAL SUPPORT METHOD

(71) Applicant: OLYMPUS MEDICAL SYSTEMS CORP., Tokyo (JP)

(72) Inventors: Kazuyoshi TAMURA, Tokyo (JP); Haruhiko SAKAYORI, Tokyo (JP); Tatsuya HONOKI, Tokyo (JP)

(73) Assignee: OLYMPUS MEDICAL SYSTEMS CORP., Tokyo (JP)

(21) Appl. No.: 18/367,600

(22) Filed: Sep. 13, 2023

Related U.S. Application Data
(63) Continuation of application No. PCT/JP2021/010506, filed on Mar. 16, 2021.

(51) Int. Cl. A61B 1/00; G06F 3/04845; G16H 40/63; G16H 30/20; G06F 3/04842

(52) U.S. Cl. CPC A61B 1/0005; G06F 3/04845; G16H 40/63; G16H 30/20; G06F 3/04842; G09G 2340/045; G09G 2380/08; G09G 2340/14

(57) ABSTRACT

An image acquisition unit acquires a simultaneously captured image group including a plurality of endoscopic images in which the same subject is imaged in a simultaneous imaging mode, and at least one of an imaging method or an image processing method is different from each other. A display image generator generates a display image and causes the display device to display the display image. The display image generator can change the number of endoscopic images to be displayed from the simultaneously captured image group.

### CROSS-REFERENCE TO RELATED APPLICATIONS

This application is based upon and claims the benefit of priority from the International Application No. PCT/JP2021/010506, filed on Mar. 16, 2021, the entire contents of which is incorporated herein by reference.

### BACKGROUND

### 1. Field of the Disclosure

The present disclosure relates to a medical support system and a medical support method.

### 2. Description of the Related Art

An endoscope observation device is connected to an endoscope inserted into a digestive tract of a patient, and displays an image of the digestive tract captured by the endoscope on a display device in real time. When a physician operates a release switch of the endoscope, the endoscope observation device captures an endoscopic image at a time when the release switch is operated, and transmits the captured endoscopic image to an image storage server. The endoscopic image stored in the image storage server is used for preparation of an examination report by the physician after the examination is completed.

JP 2008-43604 A discloses an endoscope device that simultaneously displays a normal light observation image and a narrow band light observation image obtained by imaging the same biomedical tissue on a display device in real time during an examination.

### SUMMARY

After the examination is completed, the physician operates an information processing device such as a personal computer to read the endoscopic image from the image storage server and cause the display device to display the endoscopic image in order to prepare an examination report. At this time, by displaying the endoscopic image so that the physician can easily check status of a lesion, efficient diagnosis by the physician becomes possible. The present disclosure has been made in view of such a situation, and an object thereof is to provide display technology of an endoscopic image for realizing efficient diagnosis by a physician.

A medical support system according to one aspect of the present disclosure includes: an image acquisition unit that acquires a simultaneously captured image group including a plurality of endoscopic images in which the same subject is imaged in a simultaneous imaging mode, and at least one of an imaging method or an image processing method is different from each other; and a display image generator that generates a display image and causes a display device to display the display image. The display image generator can change the number of endoscopic images to be displayed from the simultaneously captured image group.

A medical support method according to another aspect of the present disclosure includes: acquiring a simultaneously captured image group including a plurality of endoscopic images in which the same subject is imaged in a simultaneous imaging mode, and at least one of an imaging method or an image processing method is different from each other; setting the number of endoscopic images to be displayed from the simultaneously captured image group; and selecting the set number of endoscopic images from the simultaneously captured image group and causing a display device to display the endoscopic images.

Note that, arbitrary combinations of the above components and modifications of the expressions of the present disclosure among methods, devices, systems, recording media, computer programs, and the like are also effective as aspects of the present disclosure.

### BRIEF DESCRIPTION OF THE DRAWINGS

FIG. 1 is a diagram illustrating a configuration of a medical support system.

FIG. 2 is a diagram illustrating functional blocks of an information processing device.

FIG. 3 is a diagram illustrating an example of a report input screen.

FIG. 4 is a diagram illustrating an example of a list screen of endoscopic images.

FIG. 5 is a diagram illustrating an image management table.

FIG. 6 is a diagram illustrating an example of a display priority order table.

FIG. 7 is a diagram illustrating an example of a list screen of endoscopic images.

FIG. 8 is a diagram illustrating another example of the list screen of endoscopic images.

FIG. 9 is a diagram illustrating another example of the list screen of endoscopic images.

FIG. 10 is a diagram illustrating an example of an edit screen of endoscopic images.

FIG. 11 is a diagram illustrating another example of the edit screen of endoscopic images.

FIG. 12 is a diagram illustrating another example of the edit screen of endoscopic images.

FIG. 13 is a diagram illustrating a state in which a drawing mark is arranged in endoscopic images.

FIG. 14 is a diagram illustrating an example of a comparison display area.

FIGS. 15A-15C are diagrams illustrating examples of a rank table.

FIG. 16 is a diagram illustrating an example of a correspondence table.

### DETAILED DESCRIPTION

The disclosure will now be described by reference to the preferred embodiments. This does not intend to limit the scope of the present disclosure, but to exemplify the disclosure.

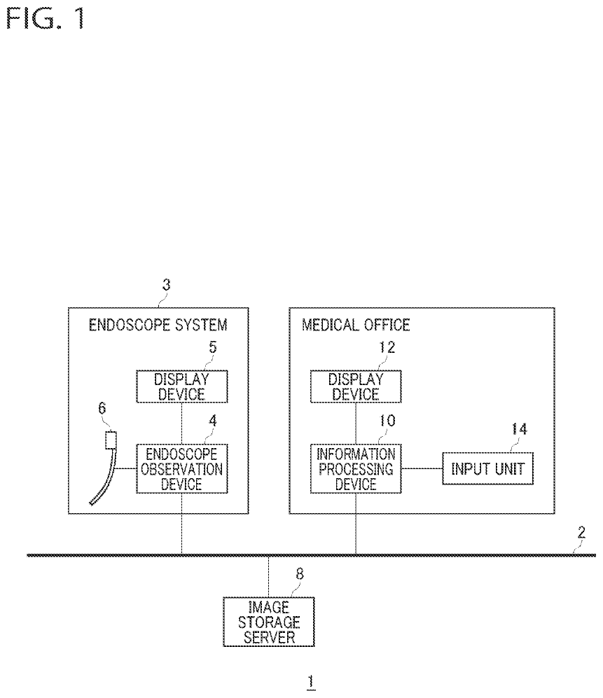

FIG. 1 illustrates a configuration of a medical support system 1 according to an embodiment. The medical support system 1 is provided in a medical facility such as a hospital that performs an endoscopic examination. In the medical support system 1, an endoscope observation device 4, an image storage server 8, and an information processing device 10 are communicably connected via a network 2 such as a local area network (LAN).

An endoscope system 3 is provided in an examination room and includes the endoscope observation device 4, a display device 5, and an endoscope 6. The endoscope 6 includes a light guide for transmitting illumination light supplied from the endoscope observation device 4 to illuminate an inside of an object under examination, and an endoscope distal end portion is provided with an illumination window for emitting the illumination light transmitted by the light guide to a subject and an imaging unit that images the subject at a predetermined cycle and outputs imaging signals to the endoscope observation device 4. The endoscope observation device 4 supplies illumination light according to an observation mode to the endoscope 6. The imaging unit includes a solid-state imaging element (for example, a CCD image sensor or a CMOS image sensor) that converts incident light into an electric signal.

The endoscope observation device 4 includes an image processor that performs image processing on the imaging signals photoelectrically converted by the solid-state imaging element, and the image processor generates an endoscopic image and outputs the endoscopic image from the display device 5. The image processor has a function of performing special image processing for a purpose of highlighting in addition to normal image processing such as A/D conversion and noise removal. Since the image processor has a special image processing function, the endoscope observation device 4 can generate an endoscopic image that has not been subjected to the special image processing and an endoscopic image that has been subjected to the special image processing from the imaging signals captured using the same illumination light. The endoscope observation device 4 is connected to the endoscope 6 inserted into the object under examination, and displays an image of the digestive tract captured by the endoscope 6 on the display device 5 in real time.

The physician observes the endoscopic image displayed on the display device 5 according to an examination procedure. When the physician operates a release switch of the endoscope 6, the endoscope observation device 4 captures (stores) the endoscopic image at a time when the release switch is operated, and transmits and records the captured endoscopic image to the image storage server 8.

### Observation Mode

The endoscope system 3 has a function of generating an endoscopic image in a plurality of observation modes, and the display device 5 displays the endoscopic image generated in one observation mode. The “observation mode” is defined by a combination of an imaging method of a subject and an image processing method of imaging signals, and the endoscope observation device 4 generates an endoscopic image in the set observation mode and displays the endoscopic image on the display device 5. The endoscope system 3 according to the embodiment has the following observation modes.

(1) White Light Imaging (WLI) Observation Mode

WLI observation mode is an observation mode in which the endoscope 6 emits normal light (white light) to image a subject, and the endoscope observation device 4 performs the normal image processing such as noise removal on imaging signals to generate an endoscopic image, and displays the endoscopic image on the display device 5. In the WLI observation mode, an endoscopic image is generated using an imaging method with normal light and a normal image processing method.

(2) Texture and Color Enhancement Imaging (TXI) Observation Mode

TXI observation mode is an observation mode in which the endoscope 6 emits normal light (white light) to image a subject, the endoscope observation device 4 performs the normal image processing such as noise removal on imaging signals, and then performs the special image processing that optimizes three elements of “structure”, “color tone”, and “brightness” of a mucosal surface to generate an endoscopic image, and displays the endoscopic image on the display device 5. In the TXI observation mode, an endoscopic image is generated using the imaging method with normal light, the normal image processing method, and a special image processing method. The TXI observation mode provides an endoscopic image in which slight changes in color tone or structural are highlighted.

(3) Red Dichromatic Imaging (RDI) Observation Mode

RDI observation mode is an observation mode in which the endoscope 6 emits narrow band light of green, amber, and red to image a subject, and the endoscope observation device 4 performs the normal image processing such as noise removal on imaging signals to generate an endoscopic image, and displays the endoscopic image on the display device 5. In the RDI observation mode, an endoscopic image is generated by using an imaging method with narrow band light of green, amber, and red and the normal image processing method. The RDI observation mode provides an endoscopic image in which a contrast of deep tissue is formed.

(4) Narrow Band Imaging (NBI) Observation Mode

NBI observation mode is an observation mode in which the endoscope 6 emits narrow band light of blue and green to image a subject, and the endoscope observation device 4 performs the normal image processing such as noise removal on imaging signals to generate an endoscopic image, and displays the endoscopic image on the display device 5. In the NBI observation mode, an endoscopic image is generated by using an imaging method with blue and green narrow band light and the normal image processing method. The NBI observation mode provides an endoscopic image in which capillaries and microstructures in a mucosal surface layer is highlighted.

(5) Autofluorescence Imaging (AFI) Observation Mode

AFI observation mode is an observation mode in which the endoscope 6 emits excitation light (390-470 nm) to image a subject, the endoscope observation device 4 performs the normal image processing such as noise removal on imaging signals, then generates an endoscopic image converted into green according to signal intensity, and displays the endoscopic image on the display device 5. In the AFI observation mode, an endoscopic image is generated using an imaging method with blue excitation light, the normal image processing method, and the special image processing method. The AFI observation mode provides an endoscopic image in which neoplastic lesions and normal mucosa are highlighted in different color tones.

As described above, the plurality of observation modes are different from each other in at least one of the imaging method or the image processing method. Specifically, when the WLI observation mode and the TXI observation mode are compared, both imaging methods use white light and are the same, but the image processing methods are different. Further, when the WLI observation mode (TXI observation mode), the RDI observation mode, the NBI observation mode, and the AFI observation mode are compared, different imaging methods (irradiation light) are used. The physician selects an observation mode suitable for an observation situation and causes the display device 5 to display the endoscopic image.

### Imaging (Capturing) Mode

The endoscope system 3 has two imaging (capturing) modes. The imaging mode is a mode in which an endoscopic image is captured in response to an operation of the release switch of the endoscope 6, and includes a “normal imaging mode” and a “simultaneous imaging mode”. During the examination, the endoscope system 3 sets either the “normal imaging mode” or the “simultaneous imaging mode” as the imaging mode.

In the “normal imaging mode”, when the release switch is operated, the endoscope system 3 captures an endoscopic image generated in the set observation mode, that is, an endoscopic image displayed on the display device 5, and transmits the endoscopic image to the image storage server 8. A capture function in the normal imaging mode is a standard function that is also installed in conventional endoscope systems.

In the “simultaneous imaging mode”, when the release switch is operated, the endoscope system 3 captures an endoscopic image generated in the set observation mode, immediately generates and captures an endoscopic image by an imaging method and an image processing method in another observation mode, and transmits a plurality of types of endoscopic images to the image storage server 8. As described above, the capture function in the simultaneous imaging mode is a function of simultaneously imaging (capturing) a plurality of endoscopic images in which at least one of the imaging method or the image processing method is different from each other, triggered by one operation of the release switch by the physician. The plurality of endoscopic images captured simultaneously are obtained by imaging substantially the same area (range) of the same subject at the same angle of view. Note that, “simultaneous” here includes substantially simultaneous, and is used as an expression including a time difference that can be regarded as substantially simultaneous. Further, the expression that the angles of view are “the same” includes that the angles of view are substantially the same, and is used as an expression including an angle error that can be regarded as substantially the same.

In the simultaneous imaging mode, the endoscope system 3 captures an endoscopic image generated in the set observation mode, and then changes at least one of the imaging method or the image processing method, and generates an endoscopic image in another observation mode. A time period for generating an endoscopic image of another observation mode is, for example, several milliseconds to several tens of milliseconds, and the endoscope observation device 4 may continue to display a still image immediately before switching the observation mode on the display device 5 while generating an endoscopic image of the switched observation mode.

Hereinafter, in the simultaneous imaging mode, an endoscopic image generated in the set observation mode is referred to as a “reference image”, and an endoscopic image generated in an observation mode other than the set observation mode is referred to as an “additional image”. In which observation mode the additional image is generated may be determined in advance in the endoscope observation device 4 in relation to the observation mode of the reference image. For example, in a case where the reference image is generated in the WLI observation mode, it may be determined that the additional image is generated in the TXI observation mode.

The observation mode of the additional image may be determined according to a part being imaged, or may be designated by the physician at the time of operating the release switch. Note that, the number of additional images is not limited to one, and a plurality of additional images may be captured by one operation of the release switch.

The endoscope observation device 4 adds metadata to the captured endoscopic image. The metadata includes at least an image number indicating an order of being captured in the examination, irradiation light information indicating a type of the imaging method, image processing information indicating whether or not the special image processing has been performed, and simultaneous imaging information indicating whether or not the image is an additional image captured in the simultaneous imaging mode. The simultaneous imaging information may include the image number of the reference image.

The image storage server 8 records the endoscopic image transmitted from the endoscope observation device 4 in association with an examination ID that identifies the endoscopic examination. When receiving a readout request for an endoscopic image designating the examination ID from the information processing device 10, the image storage server 8 transmits a plurality of endoscopic images associated with the examination ID to the information processing device 10.

The information processing device 10 is provided in a medical office and is used when a physician prepares an examination report. An input unit 14 is a tool for a user to input an operation, such as a mouse, a stylus pen, or a keyboard. When preparing an examination report, the physician causes a display device 12 to display the endoscopic image captured in the examination, checks status of a lesion, and inputs an examination result.

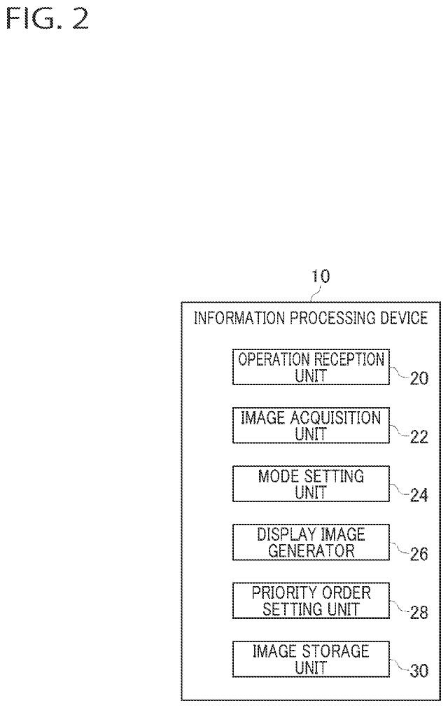

FIG. 2 illustrates functional blocks of the information processing device 10. The information processing device 10 includes an operation reception unit 20, an image acquisition unit 22, a mode setting unit 24, a display image generator 26, a priority order setting unit 28, and an image storage unit 30. The display image generator 26 generates a display image and causes the display device 12 to display the display image.

The information processing device 10 shown in FIG. 2 includes a computer, and various functions shown in FIG. 2 are implemented by the computer executing programs. The computer includes, as hardware, a memory for loading programs, one or more processors for executing the loaded programs, an auxiliary storage device, other LSIs, and the like. The processor is composed of a plurality of electronic circuits including a semiconductor integrated circuit and an LSI, and the plurality of electronic circuits may be mounted on one chip or may be mounted on a plurality of chips. It should be understood by those skilled in the art that the functional blocks shown in FIG. 2 are implemented by cooperation of hardware and software, and therefore, these functional blocks can be implemented in various forms by only hardware, only software, or a combination thereof.

After the endoscopic examination is completed, the user who is a physician enters a user ID and password to the information processing device 10 and logs in. When the user logs in, an application that prepares an examination report is activated, and a list of performed examination is displayed on the display device 12. In this performed examination list, examination information such as a patient name, a patient ID, an examination date and time, and an examination type is displayed in a list, and the user operates the input unit 14 to select an examination for which a report is to be prepared. When the operation reception unit 20 receives an examination selection operation, the image acquisition unit 22 acquires a plurality of endoscopic images associated with the examination ID of the selected examination from the image storage server 8 and stores the endoscopic images in the image storage unit 30, and the display image generator 26 generates a report input image and causes the display device 12 to display the report input image.

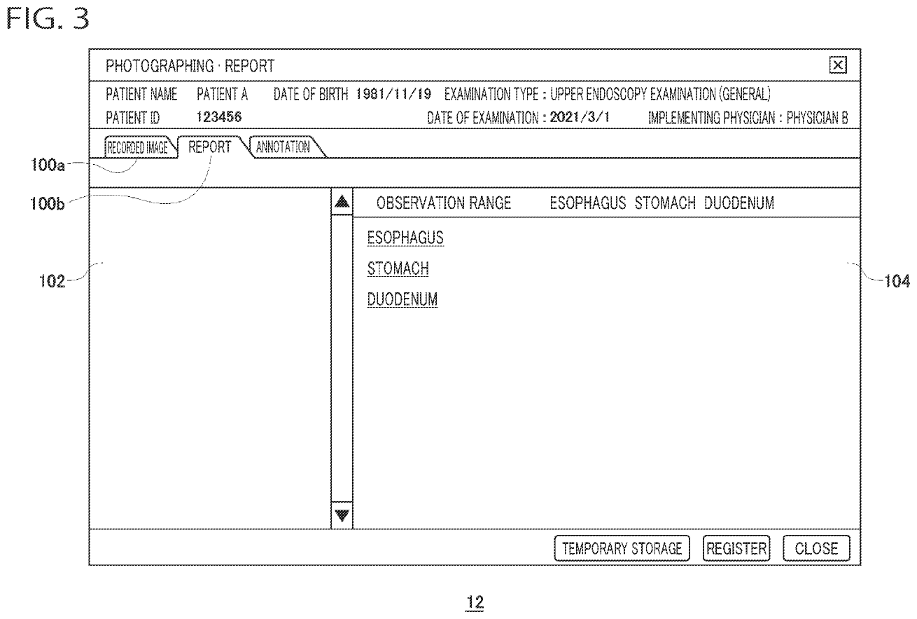

FIG. 3 illustrates an example of a report input screen. The display image generator 26 generates a report input image for the user to input the examination result, and causes the display device 12 to display the report input image. While the report input screen is displayed, a report tab 100b is selected. In an upper part of the report input screen, patient name, patient ID, date of birth, examination type, date of examination, and information of implementing physician are displayed. The report input screen includes two areas, an image display area 102 in which a thumbnail of an endoscopic image to be attached to the report is displayed is provided on a left side, and an examination result input area 104 in which the user inputs the examination result such as diagnostic content is provided on a right side.

The examination result input area 104 is an area for the user to input the examination result, and in the illustrated example, an area for inputting the diagnosis contents of “esophagus”, “stomach”, and “duodenum”, being observation ranges in an upper endoscopic examination, is provided. The examination result input area 104 may have a format in which a plurality of options of the examination result are displayed, and the diagnostic content is input by the user selecting a check box, but may have a free format for free text input.

The image display area 102 is an area for displaying the endoscopic images to be attached to the report side by side. The user selects an endoscopic image to be attached to the report from a list screen of endoscopic images. The user selects a recorded image tab 100a and displays a list of the endoscopic images captured in the examination on the display device 12.

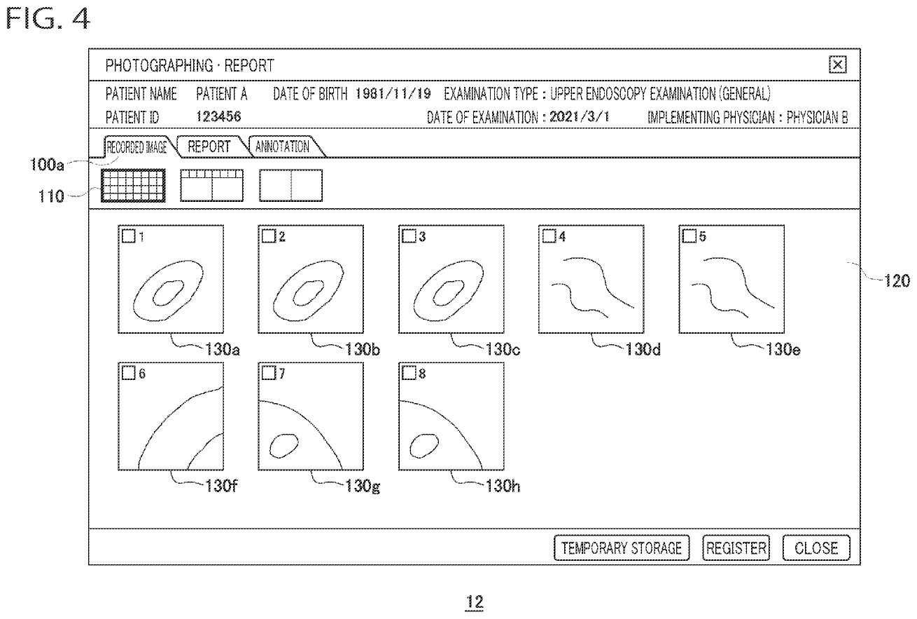

FIG. 4 illustrates an example of a list screen of endoscopic images. A list display icon 110 is an icon indicating a display format in which a list of endoscopic images is displayed. In this list screen, all endoscopic images 130a to 130h captured in the examination are displayed side by side in the image display area 120 according to the order of being captured. Note that, although it is shown a case where the number of endoscopic images captured in the examination is eight, several tens of endoscopic images are captured in an actual examination.

In the endoscope system 3 according to the embodiment, a plurality of endoscopic images are captured in which the same subject is imaged by one operation of the release switch in the simultaneous imaging mode, and at least one of the imaging method or the image processing method is different from each other. When the plurality of endoscopic images simultaneously captured in the simultaneous imaging mode are referred to as a “simultaneously captured image group”, during preparation of an examination report, the image acquisition unit 22 acquires a plurality of simultaneously captured image groups imaged at different time from the image storage server 8. For that reason, when all the endoscopic images 130a to 130h are displayed side by side, the endoscopic images included in the simultaneously captured image group are continuously arranged, so that it may hinder efficient diagnosis, particularly in a case where there are many simultaneously captured image groups. In the example shown in FIG. 4, endoscopic images 130a to 130c are included in a first simultaneously captured image group, endoscopic images 130d to 130e are included in a second simultaneously captured image group, and endoscopic images 130g to 130h are included in a third simultaneously captured image group. Note that, an endoscopic image 130f is an image captured in the normal imaging mode, and does not belong to the simultaneously captured image group.

Therefore, the information processing device 10 according to the embodiment has a function that allows the user to select the number of endoscopic images to be displayed from the simultaneously captured image group. The display image generator 26 can change the number of endoscopic images to be displayed from the simultaneously captured image group according to a selection by the user.

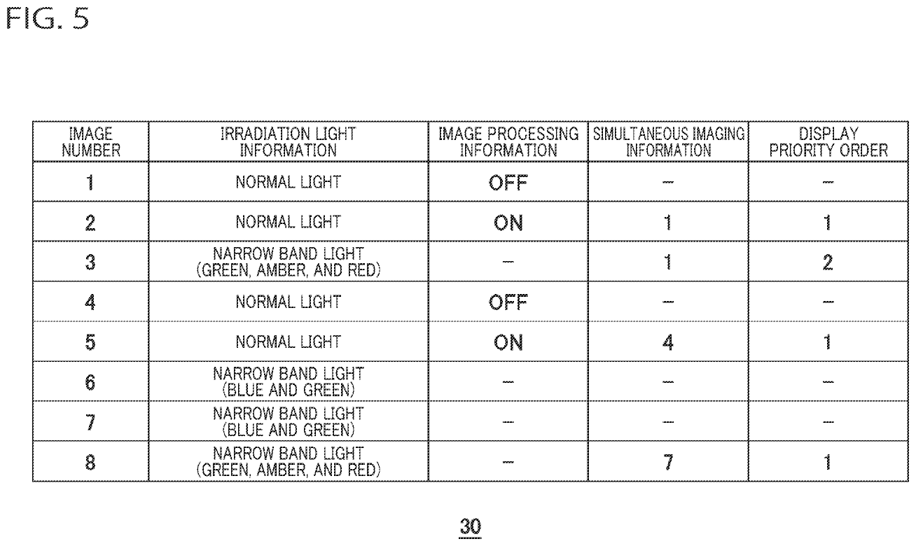

FIG. 5 illustrates an image management table held in the image storage unit 30. When storing the endoscopic image in the image storage unit 30, the image acquisition unit 22 generates an image management table from the metadata of each endoscopic image. As described above, the metadata of an endoscopic image includes the image number indicating the order of being captured, the irradiation light information indicating the type of the imaging method, the image processing information indicating whether or not the special image processing has been performed, and the simultaneous imaging information indicating whether or not the image is an additional image captured in the simultaneous imaging mode. Here, the simultaneous imaging information is the image number of the reference image.

In the image management table, the “irradiation light information” indicates the irradiation light used during imaging, and the “image processing information” indicates whether or not the special image processing has been performed. In the image processing information, item value “OFF” indicates that the special image processing has not been performed, item value “ON” indicates that the special image processing has been performed, and item value “-” indicates that there is no option to perform the special image processing on the image captured using the irradiation light. The “simultaneous imaging information” indicates whether or not the image is an additional image captured in the simultaneous imaging mode. Item value “-” indicates that the image is not an additional image, and item value “p” indicates that the image is an additional image that is simultaneously captured with the reference image of image number p. The endoscopic images of image numbers 1 to 8 correspond to the endoscopic images 130a to 130h.

The endoscopic image 130a with image number 1 is an image captured in the WLI observation mode since it is captured using normal light and has not been subjected to the special image processing.

An endoscopic image 130b with image number 2 is an image captured in the TXI observation mode since it is captured using normal light and has been subjected to the special image processing.

The endoscopic image 130c with image number 3 is an image captured in the RDI observation mode since it is captured using narrow band light (green, amber, and red). Simultaneous imaging information “1” is added to the endoscopic images 130b and 130c. Therefore, the endoscopic images 130b and 130c are images simultaneously captured as additional images for the endoscopic image 130a in the simultaneous imaging mode, and the endoscopic images 130a to 130c constitute the first simultaneously captured image group.

The endoscopic image 130d with image number 4 is an image captured in the WLI observation mode since it is captured using normal light and has not been subjected to the special image processing.

The endoscopic image 130e with image number 5 is an image captured in the TXI observation mode since it is captured using normal light and has been subjected to special image processing.

Simultaneous imaging information “4” is added to the endoscopic image 130e, and thus, the endoscopic images 130d to 130e constitute the second simultaneously captured image group.

The endoscopic image 130f with image number 6 is an image captured in the NBI observation mode since it is captured using narrow band light (blue and green). Since there is no endoscopic image to which simultaneous imaging information “6” is added in the image management table, the endoscopic image 130f is captured in the normal imaging mode.

The endoscopic image 130g with image number 7 is an image captured in the NBI observation mode since it is captured using narrow band light (blue and green).

The endoscopic image 130h with image number 8 is an image captured in the RDI observation mode since it is captured using narrow band light (green, amber, and red).

Simultaneous imaging information “7” is added to the endoscopic image 130h, and thus, the endoscopic images 130g to 130h constitute the third simultaneously captured image group.

The “display priority order” determines the order of the additional images to be preferentially displayed in a display mode for comparison and display. For example, in a case where two endoscopic images are displayed side by side in a comparison display mode, when three or more endoscopic images are included in the simultaneously captured image group, an additional image having a higher display priority order is selected and displayed side by side with the reference image.

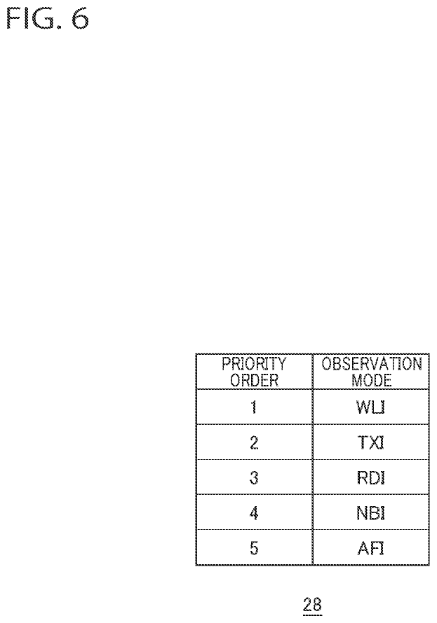

FIG. 6 illustrates an example of a display priority order table held by the priority order setting unit 28. The display priority order is set for the imaging method and/or the image processing method. In this priority order table, the order of priority display in the comparison display mode is set in the order of the observation modes of WLI, TXI, RDI, NBI, and AFI. The priority order setting unit 28 sets the priority order for the additional images of the simultaneously captured image group according to the priority order table. In the image management table shown in FIG. 5, the endoscopic images with image numbers 1 to 3 constitute a simultaneously captured image group, and priority order “1” is set to the endoscopic image with image number 2 and the priority order “2” is set to the endoscopic image with image number 3. Therefore, in a case where this simultaneously captured image group is displayed in the comparison display mode, the endoscopic image with image number 1 as a reference image and the endoscopic image with image number 2 as an additional image are displayed side by side.

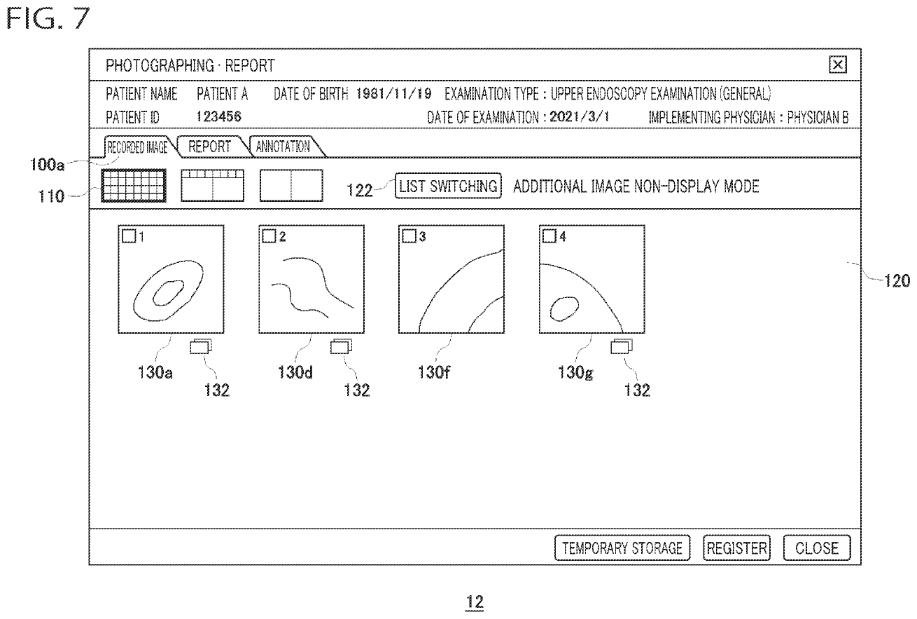

FIG. 7 illustrates an example of a list screen of the endoscopic images in the embodiment. In a list display mode, the mode setting unit 24 sets a display mode in which the number of endoscopic images to be displayed from the simultaneously captured image group is determined. The display image generator 26 generates a display image including the number of endoscopic images according to the set display mode, and causes the display device 12 to display the display image.

The list screen according to the embodiment is provided with a list switching button 122 for the user to select the number of endoscopic images to be displayed from the simultaneously captured image group. When the user operates the list switching button 122, the mode setting unit 24 switches the display mode on the list screen. In the example shown in FIG. 7, “additional image non-display mode” is written next to the list switching button 122. The additional image non-display mode is a display mode in which the number of endoscopic images to be displayed from the simultaneously captured image group is determined to be one, and when the mode setting unit 24 sets the additional image non-display mode, the display image generator 26 generates a display image including one endoscopic image selected from the simultaneously captured image group and causes the display device 12 to display the display image.

In the additional image non-display mode, the display image generator 26 selects a reference image from the simultaneously captured image group and includes the reference image in the display image. In this example, the display image generator 26 selects a reference image from the simultaneously captured image group, and each of the endoscopic images 130a, 130d, and 130g is a reference image in the simultaneously captured image group. Note that, the endoscopic image 130f is an image captured in the normal imaging mode, and does not constitute a simultaneously captured image group.

In this way, according to the additional image non-display mode, since the endoscopic images captured at different times are arranged side by side and displayed in a list, it is possible to enhance visibility of each endoscopic image and to improve diagnosis efficiency by the user. In the additional image non-display mode, the display image generator 26 arranges a simultaneous imaging icon 132 indicating presence of a non-displayed image in association with one selected endoscopic image. By displaying the simultaneous imaging icon 132, the user can recognize the presence of an endoscopic image captured in another observation mode.

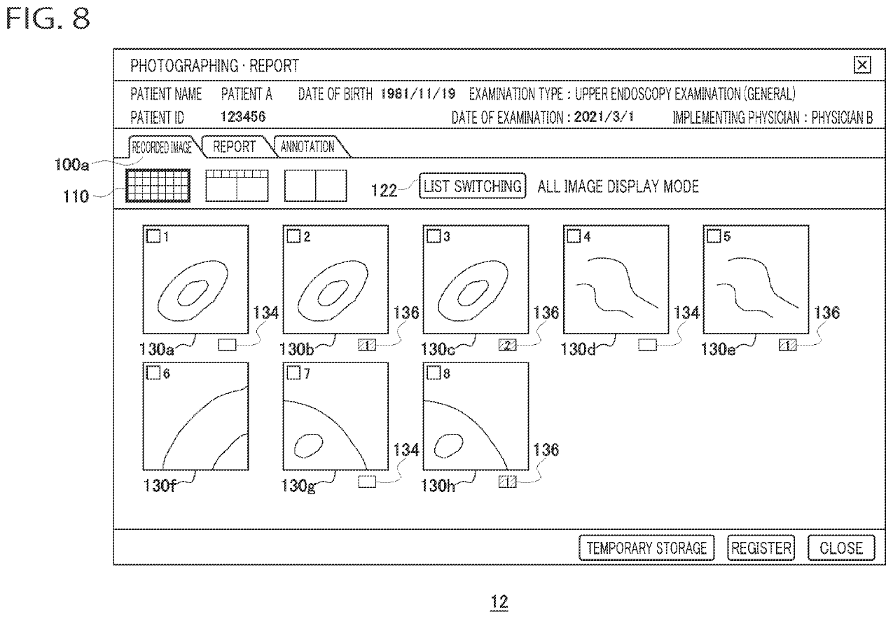

FIG. 8 illustrates another example of the list screen of the endoscopic images in the embodiment. When the user operates the list switching button 122, the mode setting unit 24 switches the display mode on the list screen. In the example shown in FIG. 8, “all image display mode” is written next to the list switching button 122. The all image display mode is a display mode in which all the endoscopic images of the simultaneously captured image group are displayed, and when the mode setting unit 24 sets the all image display mode, the display image generator 26 generates a display image including all the endoscopic images of the simultaneously captured image group and causes the display device 12 to display the display image. In the all image display mode, as on the list screen shown in FIG. 4, the endoscopic images included in the simultaneously captured image group are continuously arranged, therefore, for example, in a case where a proportion of the endoscopic images included in the simultaneously captured image group to the total number of endoscopic images is very small, the all image display mode may be set.

In the all image display mode, the display image generator 26 arranges a reference image icon 134 indicating that it is a reference image in association with the reference image of the simultaneously captured image group, and arranges an additional image icon 136 indicating that it is an additional image in association with the additional image of the simultaneously captured image group. By displaying the reference image icon 134 and the additional image icon 136, the user can recognize the simultaneously captured image group.

Note that, the display priority order in the comparison display mode may be displayed on the additional image icon 136. As described above, the priority order setting unit 28 sets the display priority order of the additional images based on the imaging method and/or the image processing method, and records the display priority order in the image management table (see FIG. 5). With reference to the image management table, the display image generator 26 includes display priority order “1” in the additional image icon 136 of the endoscopic image 130b, and includes display priority order “2” in the additional image icon 136 of the endoscopic image 130c.

The user can change the display priority order using the input unit 14. For example, when the user double-clicks the additional image icon 136 of the endoscopic image 130c using the mouse, “1” may be displayed on the additional image icon 136 of the endoscopic image 130c, and “2” may be displayed on the additional image icon 136 of the endoscopic image 130b. When the operation reception unit 20 receives a double-click operation on the additional image icon 136 of the endoscopic image 130c as a user operation of changing the display priority order, the priority order setting unit 28 may change the display priority order in the image management table. Specifically, the display priority order of image number 2 is changed to 2, and the display priority order of image number 3 is changed to 1.

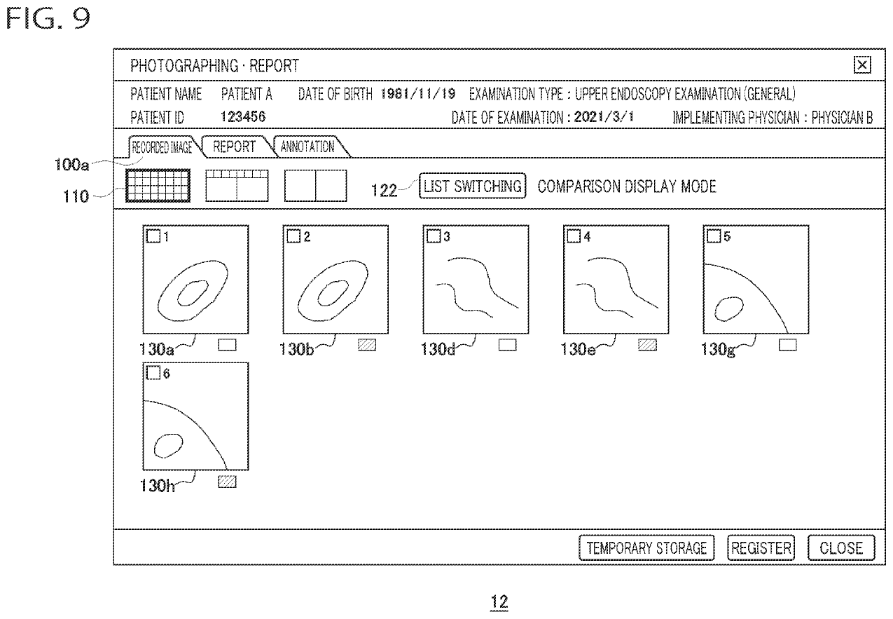

FIG. 9 illustrates another example of the list screen of the endoscopic images in the embodiment. When the user operates the list switching button 122, the mode setting unit 24 switches the display mode on the list screen. In the example shown in FIG. 9, “comparison display mode” is written next to the list switching button 122. The comparison display mode is a display mode in which the number of endoscopic images to be displayed from the simultaneously captured image group is determined to be a predetermined number of two or more. When the mode setting unit 24 sets the comparison display mode, the display image generator 26 generates a display image including the predetermined number of endoscopic images selected from the simultaneously captured image group and causes the display device 12 to display the display image. The display image generator 26 selects the predetermined number of endoscopic images from the simultaneously captured image group based on the display priority order set in the image management table. In the comparison display mode according to the embodiment, two endoscopic images are selected from the simultaneously captured image group, and displayed.

In the comparison display mode, the display image generator 26 selects a reference image and an additional image to which the display priority order “1” is set from the simultaneously captured image group, and includes the reference image and the additional image in the display image. In this example, the display image generator 26 selects the endoscopic images 130a, 130d, and 130g as the reference images and the endoscopic images 130b, 130e, and 130h to which the display priority order “1” is set, and alternately arranges the reference images and the additional images. In the comparison display mode, the endoscopic image 130c with display priority order “2” is not displayed, and the endoscopic image 130f captured in the normal imaging mode is also not displayed.

In the comparison display mode, since the additional images are displayed in a limited number as compared with the reference images, the user can efficiently compare the images with each other.

The display image generator 26 has a function of enlarging the endoscopic image 130 in an editable manner when a predetermined operation is performed on one endoscopic image 130 in the list display mode. When the operation reception unit 20 receives a user operation of instructing enlargement display of one endoscopic image 130, the display image generator 26 generates a display image including the endoscopic image 130 enlarged in an editable manner and causes the display device 12 to display the display image. The user operation of instructing enlargement display may be a double-click operation on the endoscopic image 130.

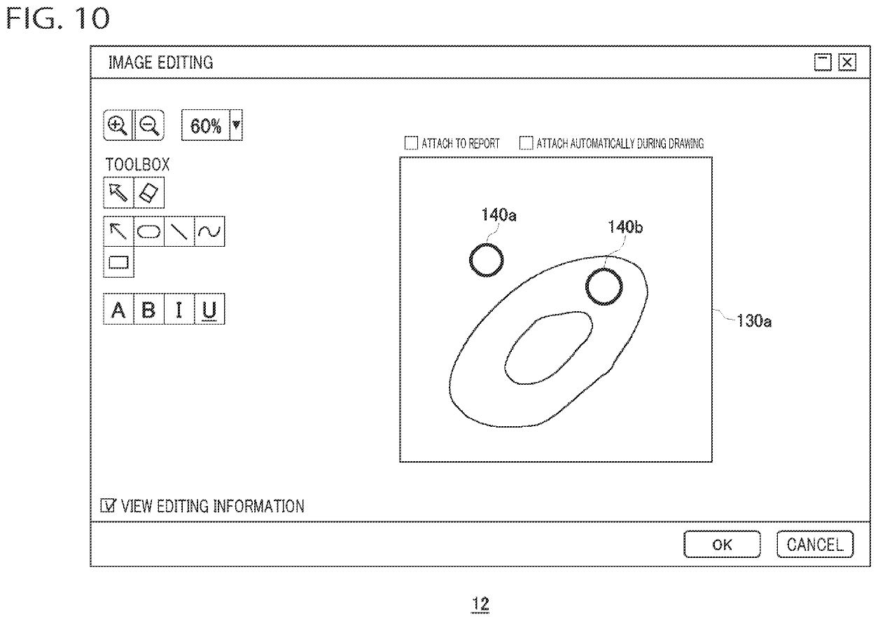

FIG. 10 illustrates an example of an edit screen of the endoscopic images in the embodiment. In the additional image non-display mode and the all image display mode, when the user performs a double-click operation on the endoscopic image 130a, the display image generator 26 generates a display image including the endoscopic image 130a enlarged in an editable manner and causes the display device 12 to display the display image. The user can perform various drawing on the endoscopic image 130a on the edit screen. In this example, the user arranges drawing marks 140a and 140b on the lesion. By attaching the edited endoscopic image to the report, a position of the lesion can be easily identified from the drawing marks 140a and 140b added to the endoscopic image later.

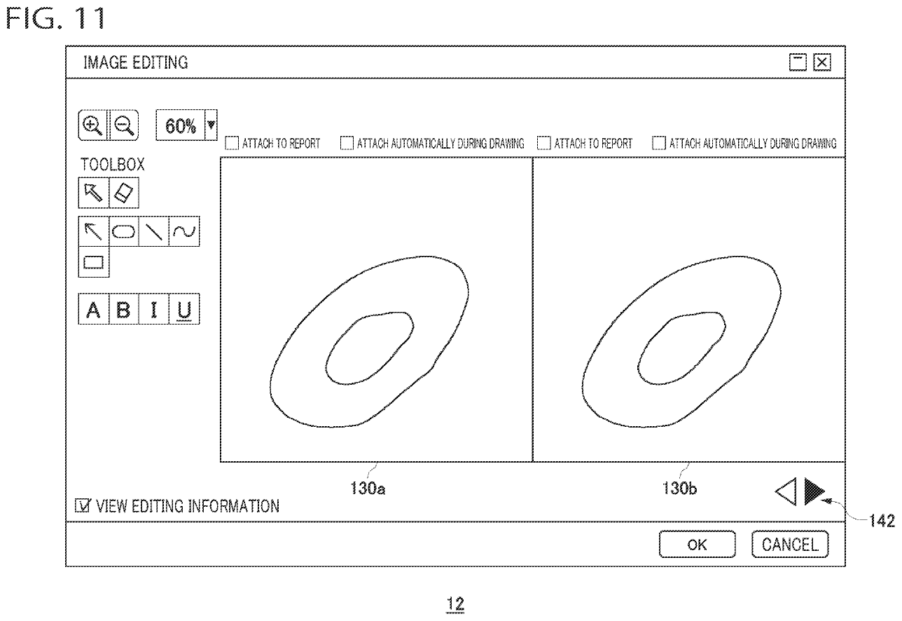

FIG. 11 illustrates another example of the edit screen of the endoscopic images in the embodiment. In the comparison display mode, when the user double-clicks the endoscopic image 130a, the display image generator 26 generates a display image including the endoscopic image 130a and the endoscopic image 130b enlarged in an editable manner and causes the display device 12 to display the display image.

In the comparison display mode, the reference image of the simultaneously captured image group and one or more additional images (one additional image in the embodiment) are always displayed as a set. Since the comparison display mode is used for the purpose that the user compares and contrasts the plurality of endoscopic images of the simultaneously captured image group, it is preferable to continue the same display manner while editing the image. Therefore, in the comparison display mode, when the operation reception unit 20 receives a double-click operation on one of the two endoscopic images selected from the simultaneously captured image group and displayed, the display image generator 26 generates a display image including the two endoscopic images and cause the display device 12 to display the display image.

In a case where the additional image to be displayed can be changed on the edit screen, the display image generator 26 arranges a change button 142 for changing the additional image on the edit screen. The case where the additional image can be changed is a case where an endoscopic image that is not displayed exists in the simultaneously captured image group. When the operation reception unit 20 receives a user operation of instructing a change in the endoscopic image, the display image generator 26 changes the enlarged endoscopic image 130b to another endoscopic image 130c in the simultaneously captured image group. Note that, in the embodiment, the image to be changed is an additional image, but it is also possible to change the reference image to an additional image.

On the edit screen, the user can change the simultaneously captured image group to be displayed. When the operation reception unit 20 receives a user operation of instructing a change in the simultaneously captured image group, the display image generator 26 generates a display image including a predetermined number of endoscopic images of different simultaneously captured image groups and causes the display device 12 to display the display image. The user operation of instructing a change in the simultaneously captured image group may be, for example, a wheel operation of the mouse.

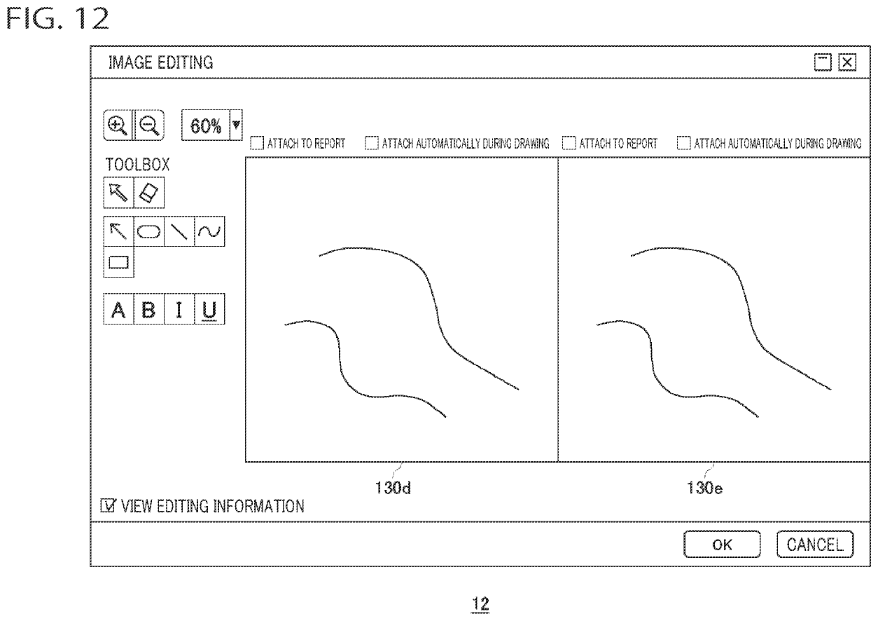

FIG. 12 illustrates another example of the edit screen of the endoscopic images in the embodiment. In a case where the comparison display mode is switched to an edit mode, it is possible to support efficient editing work of the user by continuing the display manner of the comparison display even when the simultaneously captured image group to be displayed is changed.

When the operation reception unit 20 receives a user operation of drawing on the enlarged endoscopic image, the display image generator 26 performs drawing on the endoscopic image according to the user operation and also performs drawing on another endoscopic image included in the simultaneously captured image group according to the user operation. The display image generator 26 may make the display manner of drawing different between the endoscopic image that has received the user operation and the endoscopic image that has not received the user operation.

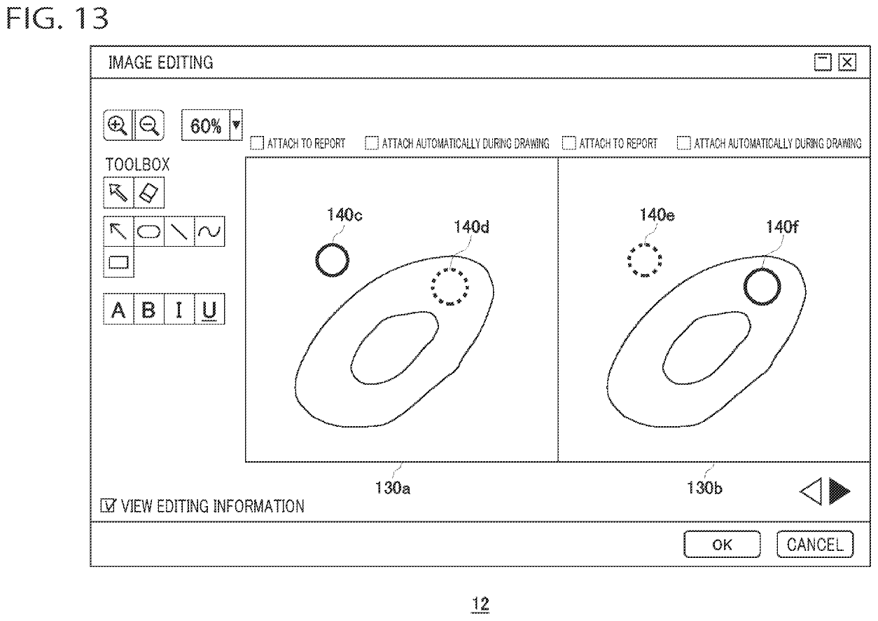

FIG. 13 illustrates a state in which the drawing mark is arranged in the endoscopic image. When drawing is performed on the endoscopic image 130a, the display image generator 26 reflects the drawing content in the endoscopic image 130b, on the other hand, when drawing is performed on the endoscopic image 130b, the display image generator reflects the drawing content in the endoscopic image 130a. Note that, processing of reflecting the drawing content is also performed on the non-displayed endoscopic image 130c in the same simultaneously captured image group. In FIG. 13, a solid line drawing mark 140c indicates the drawing content added to the endoscopic image 130a by the user, and a dotted line drawing mark 140e indicates the drawing content reflecting the drawing mark 140c. Similarly, a solid line drawing mark 140f indicates the drawing content added to the endoscopic image 130b by the user, and a dotted line drawing mark 140d indicates the drawing content reflecting the drawing mark 140f. By performing reflection processing of the drawing content in this way, it is possible to reduce trouble for the user to draw on another endoscopic image. Further, by making the display manner of drawing different between the endoscopic image that has received a drawing operation of the user and the endoscopic image that has not received the drawing operation of the user, the user can check on which endoscopic image the drawing has been performed.

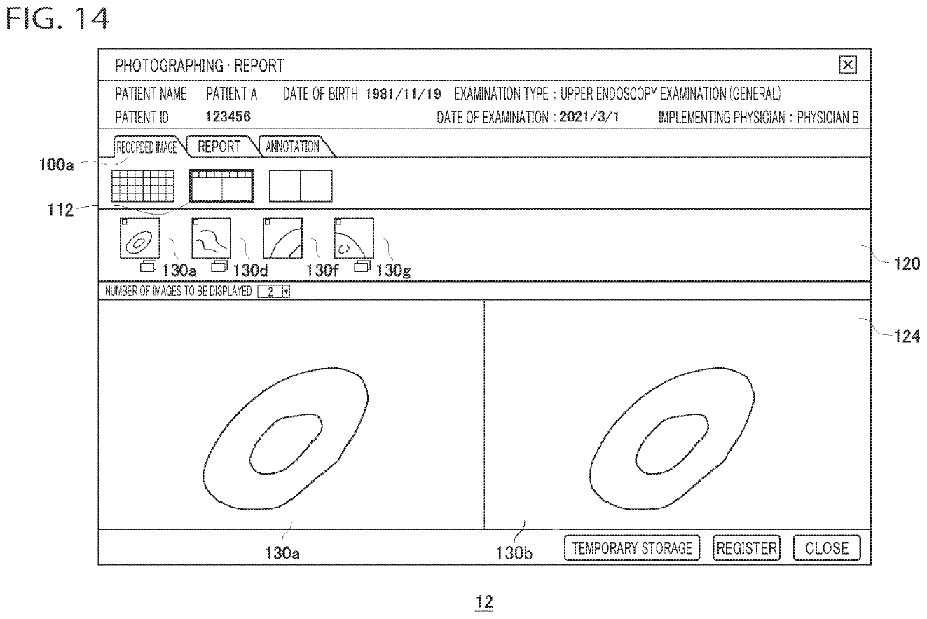

FIG. 14 illustrates an example of a comparison display area 124. When the comparison display icon 112 is selected, the display image generator 26 generates a comparison display area 124 for displaying the plurality of endoscopic images side by side, separately from the image display area 120 for displaying a list of endoscopic images. The number of images to be displayed in the comparison display area 124 may be arbitrarily selected by the user, and in this example, two images are set.

The user arranges and compares an arbitrary endoscopic image in the comparison display area 124. The user can arrange the endoscopic image in the comparison display area 124 by performing a drag and drop operation on the endoscopic image in the comparison display area 124. In the comparison display area 124, since the endoscopic image is enlarged more than a display size in the image display area 120, the image display in the comparison display area 124 is suitable for distinguishing the difference between the endoscopic images arranged by the user. The comparison display area 124 is often used, for example, when an endoscopic image captured this time is compared with an endoscopic image captured in the past.

The operation reception unit 20 receives a user's drag and drop operation of instructing display in the comparison display area 124 on the endoscopic image displayed in the image display area 120. In a case where the endoscopic image subjected to the drag and drop operation is an image included in the simultaneously captured image group, the display image generator 26 selects the set number of endoscopic images to be displayed from the simultaneously captured image group based on the display priority order, generates a display image including the comparison display area 124 in which the endoscopic images are arranged, and causes the display device 12 to display the display image. FIG. 14 illustrates a state in which the endoscopic image 130a is subjected to the drag and drop operation in the comparison display area 124, and the endoscopic image 130a and the endoscopic image 130b are displayed side by side in the comparison display area 124. Note that, when the display priority order of the endoscopic image 130c is higher than that of the endoscopic image 130b, the endoscopic image 130b and the endoscopic image 130c being included in the same simultaneously captured image group, the endoscopic image 130c is displayed next to the endoscopic image 130a.

As described above, the medical support system 1 according to the embodiment can change the number of displayed endoscopic images captured in the simultaneous imaging mode according to the display mode. Therefore, the user can efficiently perform image diagnosis by setting an appropriate display mode.

Note that, the mode setting unit 24 may automatically determine a display mode to be set first with reference to various parameters. The mode setting unit 24 may set the display mode based on at least one of an examination item, physician information, and a ratio of the number of simultaneously captured images. For example, from a viewpoint of educational purposes, a rank may be set for each of the examination item, the physician information, and the ratio of the number of simultaneously captured images, and the mode setting unit 24 may automatically set a display mode to be set first from the sum of rank values of each item.

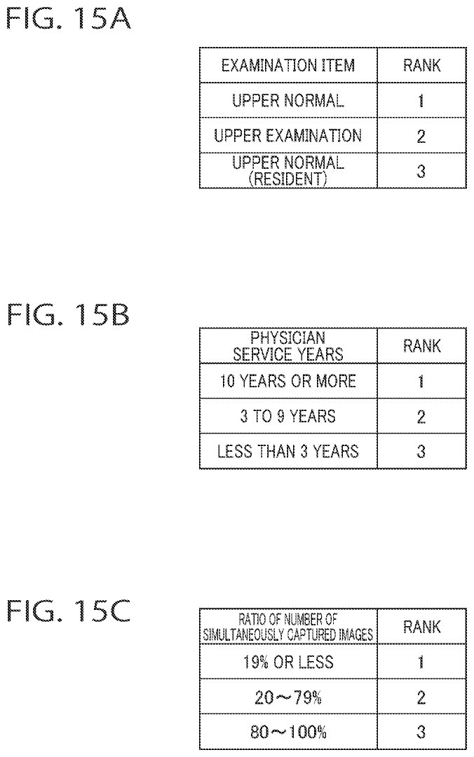

FIG. 15(a) illustrates an example of a rank table of the examination item, FIG. 15(b) illustrates an example of the rank table of physician service years, and FIG. 15(c) illustrates an example of the rank table of the ratio of the number of simultaneously captured images. These rank tables are stored in a storage device (not illustrated). The ratio of the number of simultaneously captured images is a ratio of the number of endoscopic images captured in the simultaneous imaging mode to the total number of endoscopic images captured in the examination. The mode setting unit 24 derives each rank value of the examination item, the physician information, and the ratio of the number of simultaneously captured images, and calculates a total value.

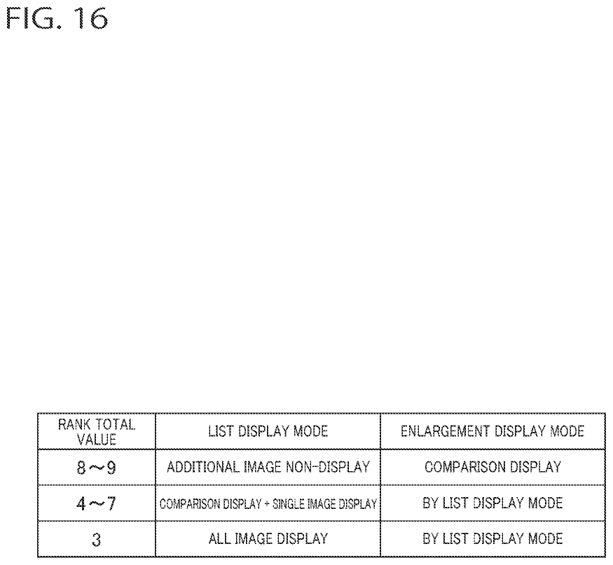

FIG. 16 illustrates an example of a correspondence table in which a rank total value and the display mode are associated with each other. This correspondence table is stored in a storage device (not illustrated). After calculating the rank total value, the mode setting unit 24 may set the list display mode and an enlargement display mode from the correspondence table. For example, it is assumed that the examination for which the rank total value is calculated to be 8 to 9 is an examination performed by a physician with little experience such as a resident, but the mode setting unit 24 sets the list display mode to the “additional image non-display mode” by default. This will allow a physician with little experience to accumulate training for diagnose using only the reference image. Note that, when the physician performs a double-click operation on the endoscopic image 130 displayed in the image display area 120, the display image generator 26 enlarges and displays two endoscopic images included in the simultaneously captured image group as shown in FIG. 11. For such a physician with little experience, in a case where the additional image non-display mode is switched to the edit mode, by displaying the endoscopic images side by side in the display manner of the comparison display mode, it is possible to contribute to improvement of diagnosis ability and efficient diagnosis of the physician.

The present disclosure has been described above based on a plurality of embodiments. It is to be understood by those skilled in the art that these embodiments are illustrative, that various modifications can be made to combinations of each component and each processing, and that such modifications are also within the scope of the present disclosure. In the embodiment, the endoscope system 3 has the “normal imaging mode” and the “simultaneous imaging mode”, but in the modification, the endoscope system 3 may have at least the “simultaneous imaging mode”, and may not have the “normal imaging mode”.

What is claimed is:

1. A medical support system comprising one or more processors having hardware, wherein
the one or more processors are configured to:
acquire a simultaneously captured image group including a plurality of endoscopic images that are simultaneously captured in response to one operation of a release switch, in which at least one of an imaging method or an image processing method is different from each other, and
select a predetermined number of endoscopic images from the simultaneously captured image group, and generate a display image that displays a list of the selected endoscopic images on a display device.

2. The medical support system according to claim 1, wherein
the one or more processors are configured to:
set the number of endoscopic images to be displayed from the simultaneously captured image group.

3. The medical support system according to claim 1, wherein
the one or more processors are configured to:
set a display mode in which the number of endoscopic images to be displayed from the simultaneously captured image group is determined, and
generate a display image including the number of endoscopic images according to the set display mode.

4. The medical support system according to claim 3, wherein
the one or more processors are configured to:
set a first display mode in which the number of endoscopic images to be displayed from the simultaneously captured image group is determined to be one, and
generate the display image including one endoscopic image selected from the simultaneously captured image group.

5. The medical support system according to claim 4, wherein
the one or more processors are configured to:
arrange an icon indicating that a non-displayed image exists in association with the selected one endoscopic image.

6. The medical support system according to claim 3, wherein
the one or more processors are configured to:
set a second display mode in which all endoscopic images of the simultaneously captured image group are displayed, and
generate the display image including all endoscopic images of the simultaneously captured image group.

7. The medical support system according to claim 6, wherein
the one or more processors are configured to:
receive a user operation of changing a display priority order that is set based on the imaging method and/or the image processing method.

8. The medical support system according to claim 3, wherein
the one or more processors are configured to:
set a third display mode in which the number of endoscopic images to be displayed from the simultaneously captured image group is determined to be a predetermined number of two or more, and
generate the display image including two or more predetermined number of endoscopic images selected from the simultaneously captured image group.

9. The medical support system according to claim 8, wherein
the one or more processors are configured to:
select two or more predetermined number of endoscopic images from the simultaneously captured image group based on the display priority order that is set based on the imaging method and/or the image processing method.

10. The medical support system according to claim 8, wherein
the one or more processors are configured to:
receive a user operation of instructing enlargement display on one of two or more predetermined number of endoscopic images selected from the simultaneously captured image group and displayed, and
generate the display image including two or more predetermined number of the enlarged endoscopic images.

11. The medical support system according to claim 10, wherein
the one or more processors are configured to:
receive a user operation of instructing a change in the endoscopic image, and
change the enlarged endoscopic image to another endoscopic image in the simultaneously captured image group.

12. The medical support system according to claim 10, wherein
the one or more processors are configured to:
acquire a plurality of the simultaneously captured image groups that are captured at different times;
receive a user operation of instructing a change in the simultaneously captured image group; and
generate the display image including a predetermined number of endoscopic images of the different simultaneously captured image groups.

13. The medical support system according to claim 10, wherein
the one or more processors are configured to:
receive a user operation of drawing on the enlarged endoscopic image, and
perform drawing according to the user operation on the endoscopic image, and also perform drawing according to the user operation on another endoscopic image included in the simultaneously captured image group.

14. The medical support system according to claim 13, wherein
the one or more processors are configured to:
make a display manner of drawing different between an endoscopic image that has received a user operation and an endoscopic image that has not received a user operation.

15. The medical support system according to claim 4, wherein
the one or more processors are configured to:
receive a user operation of instructing display of the endoscopic image displayed in a list in a comparison display area in which the number of images to be displayed is set, and
select the number of endoscopic images to be displayed from the simultaneously captured image group based on the display priority order that is set for the imaging method and/or the image processing method, and generate a display image including a comparison display area in which the endoscopic images are arranged.

16. The medical support system according to claim 3, wherein
the one or more processors are configured to:
set a display mode based on at least one of an examination item, physician information, and a ratio of the number of simultaneously captured images.

17. The medical support system according to claim 16, wherein
the one or more processors are configured to:
set a first display mode in which the number of endoscopic images to be displayed from the simultaneously captured image group is determined to be one, and
generate, from the simultaneously captured image group, a display image including an endoscopic image that is acquired by a predetermined imaging method or image processing method.

18. The medical support system according to claim 1, wherein
the plurality of endoscopic images included in the simultaneously captured image group are obtained by imaging substantially the same area.

19. The medical support system according to claim 1, wherein
the plurality of endoscopic images included in the simultaneously captured image group are acquired in an endoscope observation device triggered by one operation of the release switch by a physician.

20. A medical support method, comprising:
acquiring a simultaneously captured image group including a plurality of endoscopic images that are simultaneously captured in response to one operation of a release switch, in which at least one of an imaging method or an image processing method is different from each other,
selecting a predetermined number of endoscopic images from the simultaneously captured image group, and
generating a display image that displays a list of the selected endoscopic images on a display device.
